(19) United States
(12) Patent Application Publication
(10) Pub. No.: US 2017/0056469 A1
(43) Pub. Date: Mar. 2, 2017

(54) INDIVIDUALIZED TREATMENT OF EYE DISEASE

(71) Applicant: Mayo Foundation for Medical Education and Research, Rochester, MN (US)

(72) Inventor: Raymond Iezzi, Rochester, MN (US)

(73) Assignee: Mayo Foundation for Medical Education and Research, Rochester, MN (US)

(21) Appl. No.: 15/308,317

(22) PCT Filed: May 1, 2015
(86) PCT No.: PCT/US15/28888

§ 371 Date: Nov. 1, 2016

Related U.S. Application Data
(60) Provisional application No. 61/988,083, filed on May 2, 2014.

(51) Int. Cl. A61K 38/17; G01N 33/74; A61K 9/00; C07K 16/22; C07K 14/71

(52) U.S. Cl. CPC A61K 38/179; C07K 16/22; C07K 14/71; A61K 9/0048; G01N 33/74; C07K 2317/24; C07K 2319/30; C07K 2317/76; G01N 2333/475; G01N 2800/16; G01N 2800/52

(57) ABSTRACT

Methods are described for individualizing a patient's treatment for an eye disease that is mediated by vascular-derived endothelial growth factor.

### CROSS-REFERENCE TO RELATED APPLICATIONS

This application claims priority to U.S. Application Ser. No. 61/988,083, filed on May 2, 2014, the disclosure of which is incorporated by reference in its entirety.

### TECHNICAL FIELD

This invention relates to methods for treating patients having an eye disease. For example, this document provides methods for individualizing a patient's treatment for an eye disease that is mediated by vascular-derived endothelial growth factor.

### BACKGROUND

Over 25 million patients worldwide suffer progressive vision loss due to the physiologic effects of vascular-derived endothelial growth factor (VEGF) in eye diseases such as neovascular age-related macular degeneration (NVAMD), diabetic retinopathy, or vascular occlusive diseases of the retina. Such diseases are often treated with intraocular injections of anti-VEGF drugs, with approximately 250 million anti-VEGF injections administered each year for these conditions. While the efficacy of treatment is well established for NVAMD, many patients with diabetic retinopathy and vascular occlusive disease who receive anti-VEGF treatment do not respond, suggesting that their condition was never VEGF mediated or the dose/dosing interval was not individualized for their condition. Furthermore, patients who receive anti-VEGF injections for NVAMD often receive these injections monthly for life. To minimize injection risks and the morbidity associated with these injections, clinicians have attempted to optimize the drug dose and injection interval by using retinal imaging. Unfortunately, by the time imaging-based methods identify a need for re-treatment, disease progression has already occurred. In fact, the Comparison of Age-related macular degeneration Treatment Trials (CATT) trial showed that patients treated on an as-needed basis, guided by retinal imaging optical coherence tomography, had a worse visual acuity outcome as compared to patients who were treated monthly. See, e.g., CATT Research Group (2011). New Eng. J. Med. 364(20): 1897-1908 (2011).

### SUMMARY

This document is based, at least in part, on accurate and reproducible methods for measuring intraocular VEGF levels in a patient and tailoring the dose of an anti-VEGF drug administered to the patient and/or the interval between doses according to the needs of each individual patient. The methods and materials described herein can be used at the bedside of the patient, improving outcome and safety. For example, as described herein, an analytic technique such as an ELISA based assay or a biosensor can be used to measure VEGF and anti-VEGF drug levels in the patient's eye when therapeutic intraocular injections are administered. Such levels can be used to calculate the VEGF production rate and the clearance of the drug from the eye of the patient. The methods described herein allow individual cases to be classified according to VEGF production and provide pharmacokinetic data for individual patients, allowing an individual to receive individualized biomarker-guided treatment.

In one aspect, this document features a method for treating a patient having an eye disease. The method includes intraocularly administering a drug having affinity for VEGF to the patient; determining the clearance of the drug from the eye of the patient; determining the VEGF production rate of the patient; and adjusting the frequency of the administering step and/or adjusting the amount of the drug administered to the patient to maintain an intraocular VEGF level that is non-pathologic for that individual. The eye disease can be neovascular age-related macular degeneration (NVAMD), macular edema, diabetic macular edema (DME), a retinal vein occlusion (RVO), proliferative diabetic retinopathy (PDR), a retinal artery occlusion, an ocular ischemic syndrome, uveitis, retinitis pigmentosa, radiation retinopathy, choroidal neovascularization, neovascular glaucoma, cystoid macular edema, retinal edema, exudative retinal detachment, or central serous chorioretinopathy.

In any of the methods, the drug can be an antibody or a fragment thereof (e.g., a monoclonal antibody or fragment thereof such as Bevacizumab or Ranibizumab).

In any of the methods, the drug can be a fusion protein comprising a portion of at least one VEGF receptor that binds to VEGF (e.g., aflibercept).

In any of the methods, the clearance of the drug or VEGF product can be determined using an ELISA, mass spectrometry, electrochemiluminescence, or a biosensor.

In any of the methods, the frequency of the administering step can be adjusted and/or the amount of the drug administered to the patient can be adjusted.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims. The word “comprising” in the claims may be replaced by “consisting essentially of” or with “consisting of,” according to standard practice in patent law.

### DESCRIPTION OF DRAWINGS

FIG. 1 is a graph of anti-VEGF clearance ([AVEGF(t)], ng/mL, dashed line) and VEGF recovery ([VEGF(t)], pg/mL, solid line) after anti-VEGF delivery.

FIG. 2 is a graph of VEGF recovery after anti-VEGF suppression in humans. These data are described analytically by equation 9. See Muether et al., Am J Ophthalmol. 156:989-993 (2013) for the data.

FIG. 3 is a schematic of individualizing anti-VEGF treatment of DME by measuring VEGF levels and anti-VEGF levels with a sensor. VEGF burden refers to VEGF concentration; [anti-VEGF] refers to the concentration of the anti-VEGF drug; and trough[VEGF] refers to the lowest level of VEGF in the eye.

FIG. 4 is a schematic of individualizing anti-VEGF treatment of DME by measuring VEGF levels and anti-VEGF levels with a sensor. VEGF burden refers to VEGF concentration; [anti-VEGF] refers to the concentration of the anti-VEGF drug; and trough[VEGF] refers to the lowest level of VEGF in the eye.

### DETAILED DESCRIPTION

In general, this document features methods for treating an eye disease involving VEGF in a patient, such as neovascular age-related macular degeneration (NVAMD), macular edema, diabetic macular edema (DME), a retinal vein occlusion (RVO), proliferative diabetic retinopathy (PDR), a retinal artery occlusion, an ocular ischemic syndrome, uveitis, retinitis pigmentosa, radiation retinopathy, choroidal neovascularization, neovascular glaucoma, cystoid macular edema, retinal edema, exudative retinal detachment, or central serous chorioretinopathy. The methods include intraocularly administering a drug having affinity for VEGF to the patient, then determining the VEGF production rate after anti-VEGF treatment as well as the clearance rate of the drug having affinity for VEGF. In some cases, the methods described herein allow intraocular VEGF and anti-VEGF drug levels to be obtained in the clinic at the point of care.

Drugs having affinity for VEGF can be an antibody or a fragment thereof. For example, a drug having affinity for VEGF can be a monoclonal antibody or a fragment thereof. In some cases, the monoclonal antibody can be a humanized monoclonal antibody such as Bevacizumab (Avastin). In some cases, the monoclonal antibody fragment can be Ranibizumab (Lucentis), a humanized Fab fragment derived from the same parent antibody as Bevacizumab (Avastin). In some cases, the drug is a recombinant fusion protein that includes a portion of at least one VEGF receptor that binds to VEGF. For example, the recombinant fusion protein can be Aflibercept (Eylea), which includes VEGF-binding portions from the extracellular domains of human VEGF receptors 1 and 2 that are fused to the Fc portion of the human IgG₁ immunoglobulin.

In the methods described herein, the clearance of the drug and the VEGF production rate can be determined using any analytic methodology that can be used to measure VEGF and/or anti-VEGF drugs, including, for example, an ELISA based assay, mass spectrometry, electrochemiluminescence, or a biosensor. Intraocular VEGF or anti-VEGF drugs can be measured in aqueous fluid collected at the time of treatment. Aqueous fluid can be collected under sterile conditions using a 32 gauge needle. This poses minimal risk to the patient and is commonly performed.

After intraocularly administering anti-VEGF drug (e.g., by injecting into the vitreous cavity), drug clearance can be described by its elimination time constant, kₑ, defined by equation 1. In order to calculate this, two measurements of intraocular anti-VEGF drug concentration are made, separated by time, t.

$${k}_{e} = - \frac{{ln [ AVEGF ]}_{1} - {ln [ AVEGF ]}_{2}}{( {t}_{{[ AVEGF ]}_{1}} - {t}_{{[ AVEGF ]}_{2}} )} \\ Elimination rate constant ( 1 / min ) \qquad ( equation 1 )$$

The elimination half-life for an anti-VEGF drug then can be calculated using equation 2.

$${t}_{\frac{1}{2}} = \frac{0.693}{{k}_{e}} AVEGF Half \text{-} life ( days ) \qquad ( equation 2 )$$

Using the elimination time constant, kₑ, the dose of anti-VEGF drug administered, and the concentration of intraocular anti-VEGF drug measured at time, t after injection, the ocular volume of distribution can be calculated using equation 3.

$${V}_{d} = \frac{{Dose}_{AVEGF}}{{[ AVEGF ]}_{1}} * {e}^{- {k}_{e} {t}_{1}} \\ Volume of Distribution ( ml ) \qquad ( equation 3 )$$

The anti-VEGF initial concentration can be calculated using equation 4.

$${C}_{o} = \frac{{[ AVEGF ]}_{1}}{{e}^{- {k}_{e} {t}_{1}}} \\ AVEGF Initial Concentration ( ng / ml ) \qquad ( equation 4 )$$

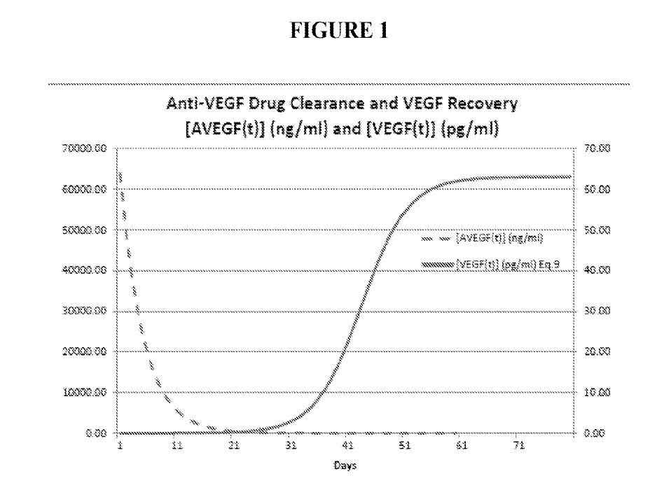

Using the anti-VEGF dose, the initial concentration, and the elimination time constant, equation 5 can be used to calculate and graph the mono-exponential decay of anti-VEGF drug concentration as a function of time (see dashed line of FIG. 1) for each patient on an individualized basis.

$$[ AVEGF ( t ) ] = \frac{D}{{V}_{d}} * {e}^{- {k}_{e} t} [ AVEGF ] after injection \qquad ( equation 5 )$$

This curve provides information as to the minimum dosing interval required to maintain some level of anti-VEGF drug in the eye. In the example provided in FIG. 1 (dashed line), re-injection with anti-VEGF drug would be required prior to 21 days in order to maintain a non-zero anti-VEGF level in the eye. This does not provide guidance, however, as to how often an individual patient requires re-injection with the anti-VEGF drug.

In order to provide guidance as to how often an individual patient requires anti-VEGF injections, the VEGF production rate and aqueous flow rate for that individual can be calculated in addition to the anti-VEGF drug clearance from the eye (dashed line in FIG. 1). Measurement of VEGF production rate is important to understanding the pharmacodynamics of anti-VEGF drug therapy. While the pharmacokinetics of anti-VEGF drugs injected into the vitreous cavity can be calculated as described above, the ocular VEGF production rate is calculated differently.

Aqueous humor flow can be calculated as this is the primary convective mechanism for drug and VEGF clearance from the eye. Because anti-VEGF drugs are not naturally occurring substances within the eye, the clearance of these drugs, after a known quantity is injected into the eye, can be used to calculate aqueous humor production and flow using equation 6.

(Clearance (ml/min) (equation 6)

Cl=kₑ*V_{d}

VEGF production rate can be calculated by measuring the steady-state VEGF concentration, [VEGF]s, in the absence of an anti-VEGF drug. Because VEGF and anti-VEGF drug elimination from the eye are highly dependent upon aqueous humor flow or clearance as described in equation six and, at steady state, VEGF production is equal to its clearance. Equation 7 describes VEGF production rate.

VEGF Production rate (pg/min) (equation 7) VEGFss(pg/ml), CL(ml/min)

VEGF_{production}=[VEGF]ₛₛ*Cl

The time required for VEGF concentration to return to a given percentage of its steady state value, [VEGF]ss, is given by equation 8. In the example given, 99% is specified. In equation 11, 75% is specified.

$$\begin{matrix}{t}_{VEGFss} = \frac{ln ( 0.01 )}{{k}_{e}} \\ Time to reach 99 \% of Vss ( days ) & ( equation 8 ) \\ {t}_{Vss} = \frac{ln ( 0.25 )}{( {k}_{e} )} \\ Time to reach 75 \% of Vss ( days ) & ( equation 10 )\end{matrix}$$

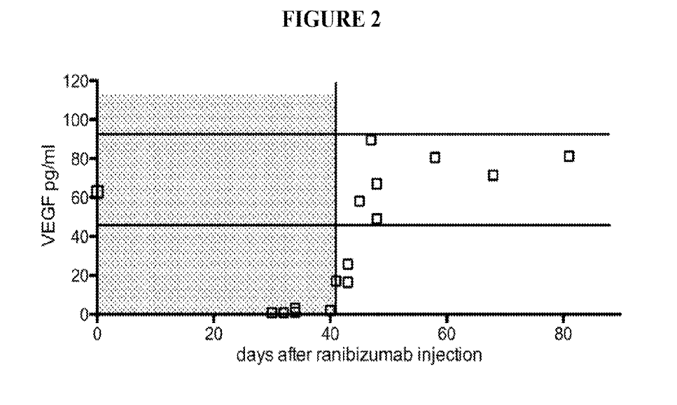

Equation 9 describes VEGF recovery after anti-VEGF suppression. It is graphed as the solid line in FIG. 1. The equation is based upon VEGF production, however simplifies so that [VEGF]ss and kₑ are required. Experimentally, this equation has been validated among patients in the clinical setting as VEGF recovery after anti-VEGF injection has been measured in many patients (see FIG. 2).

$$[ VEGF ( t ) ] = {[ VEGF ]}_{ss} * \frac{1}{1 + {e}^{- {k}_{e} ( t - ( N * {t}_{{0.5}_{AVEGF}} ) )}} \\ [ VEGF ] recovery after anti \text{-} VEGF Sigmoid \qquad ( equation 9 )$$

In some cases, equation 11 can be used to allow for five half-lives to clear the anti-VEGF drug and to allow the VEGF to return to 75% of the steady-state concentration.

$${AVEGF}_{DOSEINTERVAL} ( DAYS ) = 5 * \frac{0.693}{{k}_{e}} + \frac{ln ( 0.25 )}{{k}_{e}} \\ AVEGF dose interval to 75 \% {V}_{ss} \qquad ( equation 11 )$$

Anti-VEGF dosing interval recommendations can be based upon the time required for anti-VEGF drug to clear in addition to VEGF production rate and the time required for VEGF to return to pathologic levels for an individual. Using equation 9 or equation 11, anti-VEGF dosing can be adjusted to maintain intraocular VEGF within a pre-specified, normal, but non-pathologic range for each individual. In addition, the methods for biomarker concentration measurement (e.g., ELISA or biosensors) can be used to identify levels of VEGF and anti-VEGF drug that are safe for each individual patient. Thus, dose interval can be computed on an individualized basis, optimizing treatment.

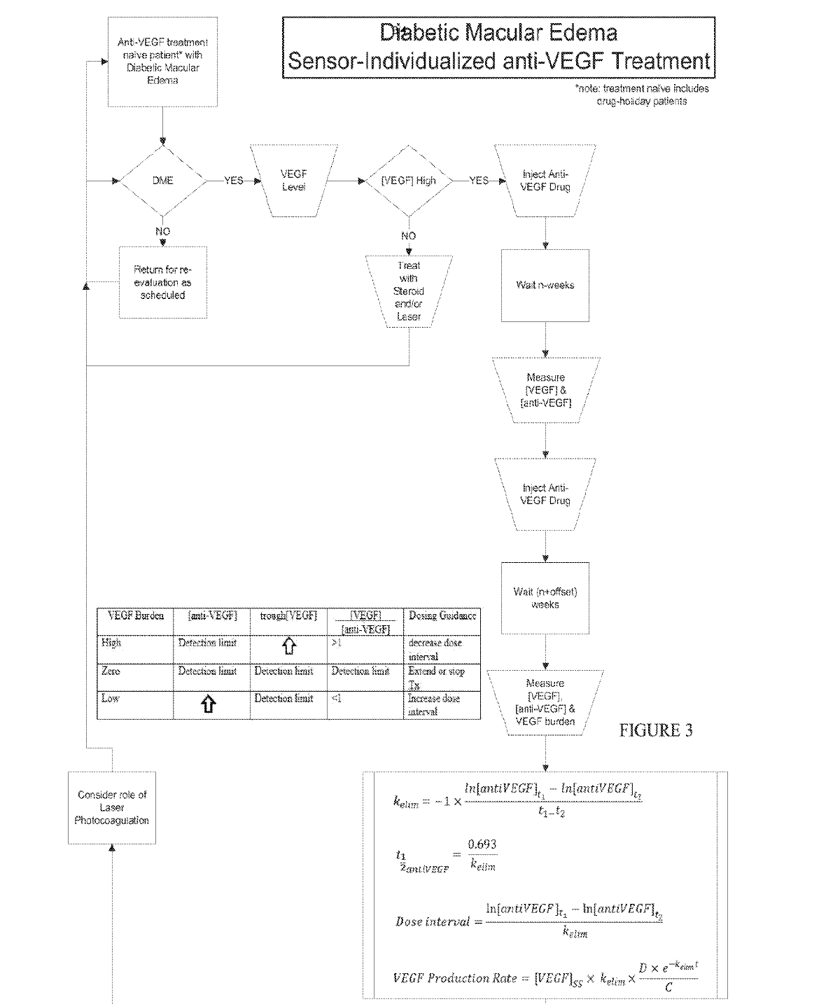

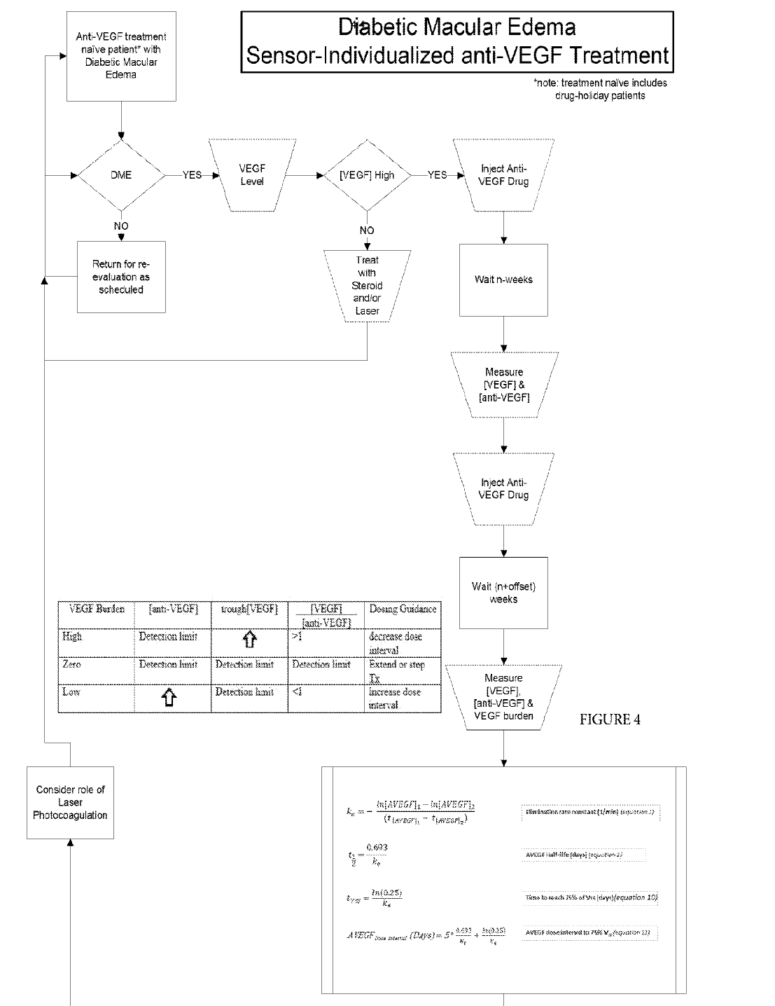

As described herein, an algorithm can be used to identify an individualized treatment plan for a person with an eye disease. For example, as shown in FIG. 3 and FIG. 4, the intraocular VEGF levels of a patient having DME can be measured (e.g., using an ELISA based assay or a sensor). If the VEGF levels are low, an anti-VEGF drug would not be administered and different treatment options could be pursued. If the VEGF levels are high, relative to non-pathologic levels of VEGF for that individual, an anti-VEGF drug can be intraocularly administered to the patient. After waiting for a period of time such as 1, 2, 3, or 4 weeks, the patient's intraocular VEGF levels can be measured again, along with the intraocular anti-VEGF drug levels. The anti-VEGF drug can be intraocularly administered again to the patient, and after waiting for a period of time such as 1, 2, 3, or 4 weeks, the intraocular levels of VEGF and the anti-VEGF drug can be measured and the clearance of the drug from the eyes of the patient and the VEGF production rate can be calculated as described above. If the VEGF burden is high and the levels of the anti-VEGF drug are low, the interval between administrations of the drug can be decreased, i.e., the frequency of administrations can be increased. If the VEGF burden is low and the levels of the anti-VEGF drug are high, the interval between administrations of the drug can be increased, i.e., the frequency of administrations is decreased. If the levels of VEGF and the level of the anti-VEGF drug are around detection limits, the interval between administrations of the drug can be increased or it may be possible to discontinue the treatment.

The invention will be further described in the following examples, which do not limit the scope of the invention described in the claims.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a patient having an eye disease, said method comprising:
a) intraocularly administering a drug having affinity for vascular-derived endothelial growth factor (VEGF) to said patient;
b) determining the clearance of said drug from the eye of said patient;
c) determining the VEGF production rate of said patient; and
d) adjusting the frequency of said administering step or adjusting the amount of said drug administered to said patient to maintain an intraocular VEGF level that is non-pathologic for that individual.

2. The method of claim 1, wherein said eye disease is neovascular age-related macular degeneration (NVAMD).

3-17. (canceled)

18. The method of claim 1, wherein said drug is an antibody or a fragment thereof.

19. The method of claim 18, wherein said antibody or fragment thereof is Bevacizumab.

20. The method of claim 18, wherein said antibody or fragment thereof is Ranibizumab.

21. The method of claim 1, wherein said drug is a fusion protein comprising a portion of at least one VEGF receptor that binds to VEGF.

22. The method of claim 21, wherein said fusion protein is aflibercept.

23. The method of claim 1, wherein the clearance of said drug is determined using an ELISA, mass spectrometry, electrochemiluminescence, or a biosensor.

24. The method of claim 1, wherein said VEGF production rate is determined using an ELISA, mass spectrometry, electrochemiluminescence, or a biosensor.

25. The method of claim 1, wherein the frequency of said administering step is adjusted.

26. The method of claim 1, wherein the amount of said drug administered to said patient is adjusted.

27. The method of claim 1, wherein both the frequency of said administering step and the amount of said drug administered to said patient is adjusted.
